(19) United States
(12) Patent Application Publication
(10) Pub. No.: US 2026/0210890 A1
(43) Pub. Date: Jul. 23, 2026

(54) Method for Testing Blood Based on Electrochemical Impedance Spectroscopy

(71) Applicant: Gwangju Institute of Science and Technology, Gwangju (KR)

(72) Inventors: Sung YANG, Gwangju (KR); Alexander ZHBANOV, Gwangju (KR); Ye Sung LEE, Gwangju (KR)

(21) Appl. No.: 19/448,790

(22) Filed: Jan. 14, 2026

(30) Foreign Application Priority Data
Jan. 14, 2025 (KR) 10-2025-0005303
Jan. 14, 2026 (KR) 10-2026-0007030

(51) Int. Cl. G01N 27/02; G01N 33/49

(52) U.S. Cl. CPC G01N 27/026; G01N 33/49

(57) ABSTRACT

An embodiment relates to a method for testing blood based on electrochemical impedance spectroscopy, the method being able to calculate clinical indicators in real time without separate clinical test equipment for calculating clinical indicators by changing initial values of blood parameters to fit an actual impedance and a virtual impedance of blood, and calculating clinical indicators using the blood parameters of which the initial values are changed.

### CROSS REFERENCE TO RELATED APPLICATIONS

The present application claims priority to Korean Patent Application No. 10-2025-0005303, filed on Jan. 14, 2025, and Korean Patent Application No. 10-2026-0007030, filed on Jan. 14, 2026, the entire contents of which are incorporated here for all purposes by this reference.

### BACKGROUND

### Technical Field

The present disclosure relates to a method for testing blood that adjusts initial values of blood parameters to fit an actual impedance and a virtual impedance of blood, and calculates clinical indicators using the blood parameters of which initial values have been adjusted.

### Description of the Related Art

Electrochemical Impedance Spectroscopy (EIS) is a technique of evaluating various physical properties of a sample, such as permittivity and electrical conductivity by applying an electrical signal to a sample such as blood and analyzing a resulting change in impedance.

However, as described above, the EIS technology of the related art is useful for extracting basic electrical properties of blood, such as permittivity and electrical conductivity, but cannot quantitatively calculate clinical indicators for blood testing, such as mean corpuscular volume (MCV), red blood cell count (RBC), mean corpuscular hemoglobin concentration (MCHC), hemoglobin concentration (Hb), hematocrit (HCT), and mean corpuscular hemoglobin (MCH), and even when separate clinical test equipment for calculating clinical indicators, such as a point-of-care test (POCT), is used, there is difficulty in calculating the clinical indicators in real time.

Accordingly, there is a need for a technology that can quantitatively calculate clinical indicators based on electrochemical impedance spectroscopy.

### SUMMARY

An objective of the present disclosure is to provide a method for testing blood that adjusts initial values of blood parameters to fit an actual impedance and a virtual impedance of blood, and calculates clinical indicators using the blood parameters of which initial values have been adjusted.

The method for testing blood based on electrochemical impedance spectroscopy according to an embodiment of the present disclosure may include: measuring an actual impedance of blood; calculating virtual impedance using initial values of a plurality of blood parameters; changing the blood parameters from the initial values to fit the virtual impedance to the actual impedance; and calculating at least one clinical indicator for the blood on the basis of the plurality of blood parameters changed from the initial values.

The plurality of blood parameters may include permittivity and electrical conductivity of hemoglobin including hydration layers, red blood cell intracellular fluid, a red blood cell membrane, and plasma, and parameters for calculating the permittivity and the electrical conductivity.

The calculating of a virtual impedance may include: calculating complex relative permittivity by inputting the plurality of blood parameters into a mathematical equation defined in accordance with a virtual blood model that reflects multiple phases in blood, volume ratios of the multiple phases, and an orientation of red blood cells; and calculating the virtual impedance on the basis of the complex relative permittivity.

The multiple phases may include at least one of intracellular fluid, hemoglobin, a primary hydration layer, a secondary hydration layer, a red blood cell membrane, and plasma.

The orientation of the red blood cells may include an aligned orientation or a random orientation.

The changing from the initial values may change the blood parameters from the initial values through an optimization algorithm by setting an objective function that uses the plurality of blood parameters as independent variables and the virtual impedance as a dependent variable and minimizes a difference between the virtual impedance and the actual impedance.

The calculating of a clinical indicator may include calculating a blood clinical indicator through a conversion equation corresponding to the clinical indicator on the basis of the blood parameters changed from the initial values.

The clinical indicator may include mean corpuscular volume (MCV), a red blood cell count (RBC), mean corpuscular hemoglobin concentration (MCHC), hemoglobin concentration (Hb), hematocrit (HCT), and mean corpuscular hemoglobin (MCH).

The present disclosure can calculate clinical indicators in real time without separate clinical test equipment by changing initial values of blood parameters to fit an actual impedance and a virtual impedance of blood, and calculating clinical indicators using the blood parameters of which the initial values are changed.

Detailed effects of the present disclosure in addition to the above effects will be described with the following detailed description for accomplishing the present disclosure.

### BRIEF DESCRIPTION OF THE DRAWINGS

FIG. 1 is a flowchart illustrating a method for testing blood based on electrochemical impedance spectroscopy;

FIG. 2 is a graph showing a Nyquist plot according to impedance characteristics;

FIG. 3 is a graph showing a comparison result between a mean corpuscular volume measured by a standard measurement method and a mean corpuscular volume calculated using parameters with adjusted initial values;

FIG. 4 is a graph showing clinical indicators calculated using a standard measurement method and parameters with adjusted initial values;

FIG. 5 is a graph showing a comparison result of hematocrit;

FIG. 6 is a graph showing a comparison result of mean corpuscular hemoglobin;

FIG. 7 is a graph showing a comparison result of a red blood cell count;

FIG. 8 is a graph showing a comparison result of mean corpuscular hemoglobin concentration; and

FIG. 9 is a graph showing a comparison result of hemoglobin concentration.

### DETAILED DESCRIPTION

The objectives, characteristics, and advantages will be described in detail below with reference to the accompanying drawings, so those skilled in the art may easily achieve the spirit of the present disclosure. However, in describing the present disclosure, detailed descriptions of well-known technologies will be omitted so as not to obscure the description of the present disclosure with unnecessary details. Hereinafter, exemplary embodiments of the present disclosure are described in detail with reference to the accompanying drawings. The same reference numerals are used to indicate the same or similar components in the drawings.

Although terms “firs”, “second”, etc. are used to describe various components in the specification, it should be noted that these components are not limited by the terms. These terms are used to discriminate one component from another component and it is apparent that a first component may be a second component unless specifically stated otherwise.

Further, in the specification, when a certain configuration is disposed “over (or under)” or “on (beneath)” of a component in the following description, it may mean not only that the certain configuration is disposed on the top (or bottom) of the component, but that another configuration may be interposed between the component and the certain configuration disposed on (or beneath) the component.

Further, in the specification, when a certain component is “connected”, “coupled”, or “jointed” to another component, it should be understood that the components may be directly connected or jointed to each other, but another component may be “interposed” between the components or the components may be “connected”, “coupled”, or “jointed” through another component.

Further, singular forms that are used in this specification are intended to include plural forms unless the context clearly indicates otherwise. In this application, terms “configured”, “include”, or the like should not be construed as necessarily including several components or several steps described herein, in which some of the components or steps may not be included or additional components or steps may be further included.

Further, in this specification, the term “A and/or B” stated in the specification means that A, B, or A and B unless specifically stated otherwise, and the term “C to D” means that C or more and D or less unless specifically stated otherwise.

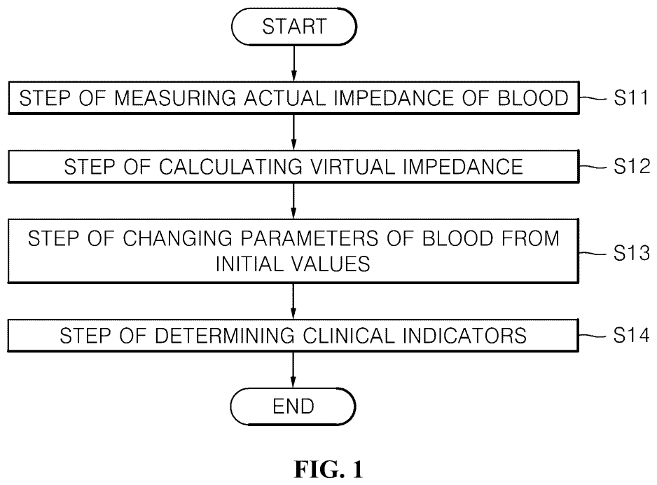

Referring to FIG. 1, a method for testing blood based on electrochemical impedance spectroscopy according to an embodiment of the present disclosure may include a step of measuring an actual impedance of blood, a step of calculating a virtual impedance, a step of adjusting initial values, and a step of determining clinical indicators.

However, the method for testing blood based on electrochemical impedance spectroscopy illustrated in FIG. 1 relates to an embodiment, and the steps constituting the present disclosure are not limited to those illustrated in FIG. 1, and some steps may be added, modified, or omitted as necessary.

the method for testing blood based on electrochemical impedance spectroscopy may be performed by a processor, and to this end, the processor may include at least one physical element of application specific integrated circuits (ASICs), digital signal processors (DSPs), digital signal processing devices (DSPDs), programmable logic devices (PLDs), field programmable gate arrays (FPGAs), a controller, micro-controllers.

The processor can measure an actual impedance of blood (S11). In this case, the actual impedance may be an impedance obtained by measuring the impedance of blood flowing through a microfluidic device. The actual impedance may be corrected by removing parasitic components generated from the microfluidic device.

To this end, the processor can measure a first impedance in a state in which blood does not flow in the microfluidic device. That is, the processor can measure the impedance of air in an empty channel of the microfluidic device. Through this, the stray capacitance (Cs) of the microfluidic device can be identified. The stray capacitance may be generated by the structure of the microfluidic device.

Subsequently, the processor can measure a second impedance of water and ultrapure water flowing through the microfluidic device. Accordingly, the processor can identify an electrode polarization component (constant phase element (CPE)). The electrode polarization component may be a component generated as ions are accumulated on an electrode surface in the microfluidic device.

Subsequently, the processor can calculate an actual impedance by correcting the impedance of blood flowing through the microfluidic device using the measured first and second impedances. Specifically, the processor can calculate the actual impedance of blood itself by removing a stray capacitance and an electrode polarization component from an equivalent circuit that includes a stray capacitance, an electrode polarization component, and a blood impedance.

Subsequently, the processor can calculate a virtual impedance using initial values of a plurality of blood parameters (S12). Specifically, the processor can calculate complex relative permittivity by inputting a plurality of blood parameters into a mathematical equation defined in accordance with a virtual blood model that reflects multiple phases in blood, the volume ratios of the multiple phases, and the orientation of red blood cells. In this case, the multiple phases may include at least one of intracellular fluid (ICF), hemoglobin, a primary hydration layer, a secondary hydration layer, a red blood cell (RBC) membrane, and plasma, and the orientation of red blood cells may be an aligned orientation or a random orientation. Meanwhile, red blood cells may be assumed to be ellipsoids.

The plurality of blood parameters may include permittivity and electrical conductivity of hemoglobin including hydration layers, red blood cell intracellular fluid, red blood cells, and plasma, and parameters for calculating the permittivity and the electrical conductivity.

To further describe, the plurality of parameters may include permittivity and electrical conductivity of hemoglobin including primary and secondary hydration layers, permittivity and electrical conductivity of a hemoglobin core, and a diameter of hemoglobin, and may include permittivity and electrical conductivity of red blood cell intracellular fluid and a volume ratio of hemoglobin in red blood cells.

The plurality of parameters may include permittivity and electrical conductivity of red blood cells, a red blood cell volume fraction, red blood cell sizes (in x, y, z), permittivity and electrical conductivity of a red blood cell membrane, and a thickness of the red blood cell membrane, and may include permittivity and electrical conductivity of plasma.

Subsequently, the processor can calculate a virtual impedance on the basis of the complex relative permittivity. Specifically, the processor can calculate resistance (R) and capacitance (C) from the complex relative permittivity, and can derive a virtual impedance using the resistance (R) and capacitance (C). In this case, the processor may further correct the virtual impedance through equivalent circuit analysis that includes a virtual impedance, an electrode polarization component (CPE), and a stray capacitance (Cs) in accordance with correction of an actual impedance.

Subsequently, the processor can change blood parameters from the initial values in order to fit the virtual impedance to the actual impedance (S13). Specifically, the processor can change the blood parameters from the initial values through an optimization algorithm by setting an objective function that uses the plurality of blood parameters as independent variables and the virtual impedance as a dependent variable and minimizes a difference (error rate) between the virtual impedance and the actual impedance.

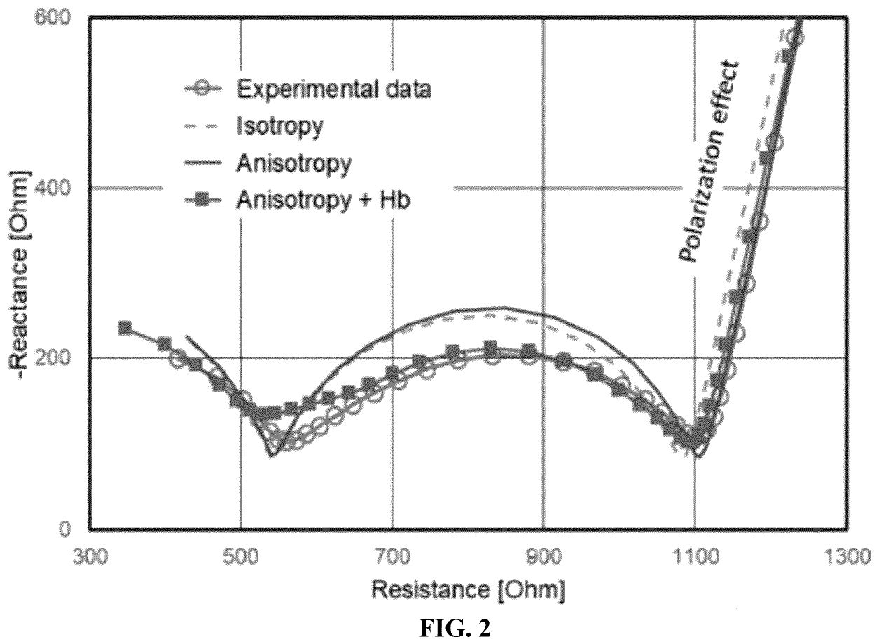

Accordingly, referring to FIG. 2, as shown in a Nyquist plot, the difference between the virtual impedance and the actual impedance is minimized, so that the virtual impedance based on initial values of virtual blood parameters (isotropy or anisotropy) can be gradually fitted to the actual impedance (experimental data), and accordingly, the virtual impedance (anisotropy+Hb) after changing the blood parameters from the initial values can match the actual impedance.

Subsequently, the processor can calculate at least one clinical indicator for blood on the basis of the plurality of blood parameters changed from initial values (S14). Specifically, the processor can calculate a blood clinical indicator through a conversion equation corresponding to the clinical indicator on the basis of the plurality of blood parameters changed from the initial values. In this case, the clinical indicator may include mean corpuscular volume (MCV), a red blood cell count (RBC), mean corpuscular hemoglobin concentration (MCHC), hemoglobin concentration (Hb), hematocrit (HCT), and mean corpuscular hemoglobin (MCH).

$$MCV = \frac{4 π {a}_{x} {a}_{y} {a}_{z}}{3} \qquad [ Equation 1 ]$$

For example, an equation for calculating a red blood cell volume may be expressed as Equation 1, and the processor can calculate the red blood cell volume by substituting red blood cell sizes (x, y, z) parameters into the conversion equation.

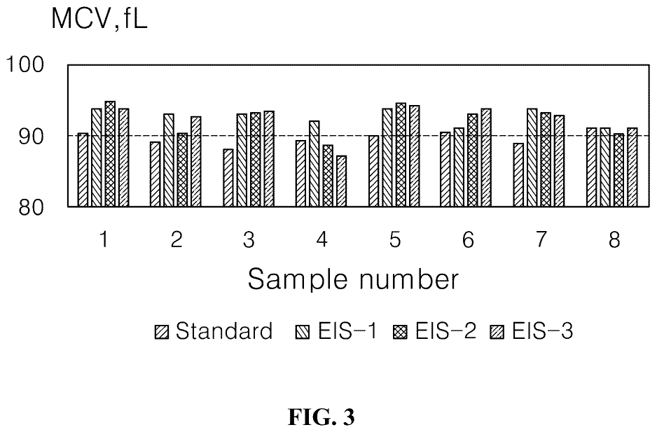

In this regard, referring to FIG. 3, a comparison of red blood cell volumes (MCV) is shown, in which a standard indicates a red blood cell volume measured by a standard method, and data 1 (EIS-1), data 2 (EIS-2), and data 3 (EIS-3) indicate red blood cell volumes calculated by applying parameters changed from initial values to conversion equations corresponding to clinical indicators.

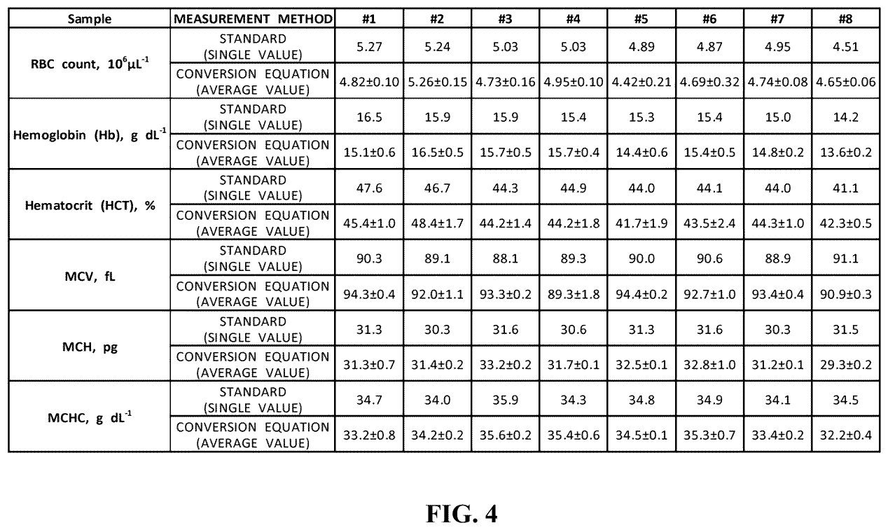

As a result of the comparison, referring to FIG. 4, it is shown that the difference between a red blood cell volume value (a single value) measured by a standard method and a red blood cell volume value (an average value) calculated through a conversion equation corresponding to a clinical indicator using parameters changed from initial values is not large, and thus reliability is shown to be verified.

$$HCT = 100 \times ϕ \qquad [ Equation 2 ]$$

To further describe, an equation for calculating hematocrit may be expressed as Equation 2, and the processor can calculate hematocrit by substituting a red blood cell volume fraction parameter into the conversion equation.

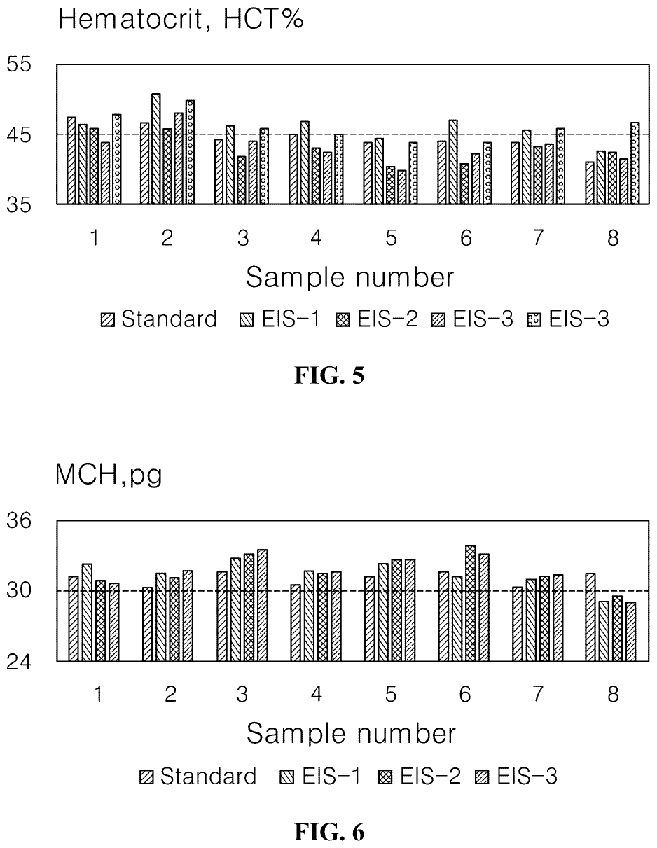

In this regard, referring to FIG. 5, a comparison of hematocrit is shown, in which a standard indicates hematocrit measured by a standard method, data 1 (EIS-1), data 2 (EIS-2), and data 3 (EIS-3) indicate hematocrit calculated by applying parameters changed from initial values to conversion equations corresponding to clinical indicators, and laboratory data (LAB) may indicate hematocrit measured by a centrifuge in a laboratory.

As a result of comparison, referring again to FIG. 4, it is shown that the difference between hematocrit (a single value, excluding hematocrit measured by a centrifuge) measured by a standard method and hematocrit (an average value) calculated through a conversion equation corresponding to a clinical indicator using parameters changed from initial values is not large, and thus reliability is shown to be verified.

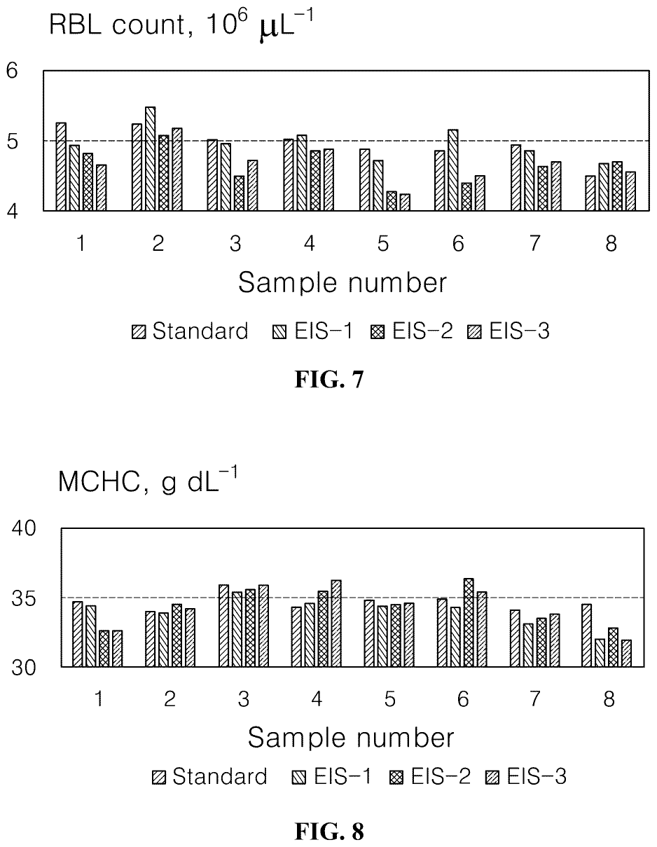

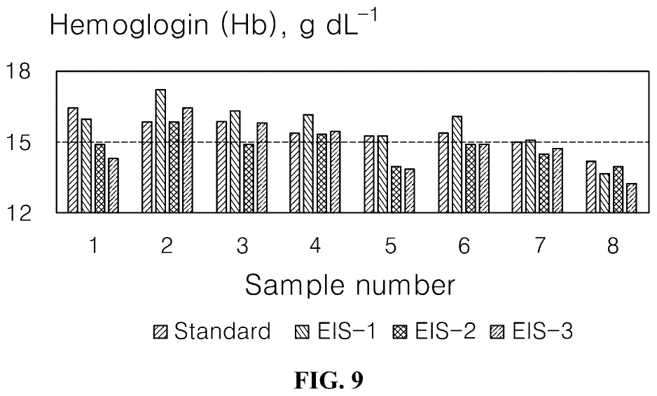

Additionally, referring to FIGS. 6 to 9 together, remaining parameters including mean corpuscular hemoglobin (MCH), a red blood cell count (RBC), mean corpuscular hemoglobin concentration (MCHC), and hemoglobin concentration (Hb) can be confirmed, in which, as described above, a standard indicates values measured by a standard method, and data 1 (EIS-1), data 2 (EIS-2), and data 3 (EIS-3) may indicate values calculated through conversion equations corresponding to clinical indicators using parameters changed from initial values.

As a result of the comparison, referring again to FIG. 4, it is shown that the difference between clinical indicators (MCV, RBC, MCHC, and Hb) measured by a standard method and clinical indicators (average values) calculated through conversion equations corresponding to the clinical indicators using parameters changed from initial values is not large, and thus, as described above, reliability is shown to be verified.

Accordingly, the present disclosure can calculate clinical indicators in real time without separate clinical test equipment by changing initial values of blood parameters to fit an actual impedance and a virtual impedance of blood, and calculating clinical indicators using the blood parameters of which the initial values are changed.

Although the present disclosure was described above with reference to the exemplary drawings, it is apparent that the present disclosure is not limited to the embodiments and drawings in the specification and may be modified in various ways by those skilled in the art within the range of the spirit of the present disclosure. Further, even though the operation effects according to the configuration of the present disclosure were not clearly described with the above description of embodiments of the present disclosure, it is apparent that effects that can be expected from the configuration should be also admitted.

What is claimed is:

1. A method for testing blood based on electrochemical impedance spectroscopy, the method comprising:
measuring an actual impedance of blood;
calculating virtual impedance using initial values of a plurality of blood parameters;
changing the blood parameters from the initial values to fit the virtual impedance to the actual impedance; and
calculating at least one clinical indicator for the blood on the basis of the plurality of blood parameters changed from the initial values.

2. The method of claim 1, wherein the plurality of blood parameters include permittivity and electrical conductivity of hemoglobin including hydration layers, red blood cell intracellular fluid, a red blood cell membrane, and plasma, and parameters for calculating the permittivity and the electrical conductivity.

3. The method of claim 1, wherein the calculating of a virtual impedance includes:
calculating complex relative permittivity by inputting the plurality of blood parameters into a mathematical equation defined in accordance with a virtual blood model that reflects multiple phases in blood, volume ratios of the multiple phases, and an orientation of red blood cells; and
calculating the virtual impedance on the basis of the complex relative permittivity.

4. The method of claim 3, wherein the multiple phases include at least one of intracellular fluid, hemoglobin, a primary hydration layer, a secondary hydration layer, a red blood cell membrane, and plasma.

5. The method of claim 3, wherein the orientation of the red blood cells includes an aligned orientation or a random orientation.

6. The method of claim 1, wherein the changing from the initial values changes the blood parameters from the initial values through an optimization algorithm by setting an objective function that uses the plurality of blood parameters as independent variables and the virtual impedance as a dependent variable and minimizes a difference between the virtual impedance and the actual impedance.

7. The method of claim 1, wherein the calculating of a clinical indicator includes calculating a blood clinical indicator through a conversion equation corresponding to the clinical indicator on the basis of the blood parameters changed from the initial values.

8. The method of claim 1, wherein the clinical indicator includes mean corpuscular volume (MCV), a red blood cell count (RBC), mean corpuscular hemoglobin concentration (MCHC), hemoglobin concentration (Hb), hematocrit (HCT), and mean corpuscular hemoglobin (MCH).
